(19) United States
(12) Patent Application Publication
(10) Pub. No.: US 2011/0091579 A1
(43) Pub. Date: Apr. 21, 2011

(54) VITAMIN D2 ENRICHED MUSHROOMS AND FUNGI FOR TREATMENT OF OXIDATIVE STRESS, ALZHEIMER'S DISEASE AND ASSOCIATED DISEASE STATES

(75) Inventor: MARVIN S. HAUSMAN, SHERWOOD, OR (US)

(73) Assignee: TOTAL NUTRACEUTICAL SOLUTIONS, INC., STEVENSON, WA (US)

(21) Appl. No.: 12/887,276

(22) Filed: Sep. 21, 2010

Related U.S. Application Data
(60) Provisional application No. 61/277,150, filed on Sep. 21, 2009.
(60) Provisional application No. 61/280,578, filed on Nov. 5, 2009.
(60) Provisional application No. 61/335,394, filed on Jan. 6, 2010.

(51) Int. Cl. A61K 33/04; A61K 36/06; A61P 25/28

(52) U.S. Cl. 424/702; 424/195.15

(57) ABSTRACT

An filamentous fungi is disclosed with a naturally vitamin D enriched nutritional profile. These enriched mushrooms were shown to have a synergistic effect on longevity of subjects with both a normal and nutritionally deficient diets, improved tolerance to oxidative stress, and increased longevity in a Alzheimer's disease model. Surprisingly, vitamin D2 and D3 fed alone or in combination with nonenriched mushrooms did not produce similar effects.

### CROSS-REFERENCE TO RELATED APPLICATIONS

This application claims priority under 35 U.S.C. §119 to provisional application Ser. Nos. 61/277,150 filed Sep. 21, 2009, 61/280,578 filed Nov. 5, 2009, and 61/335,394 filed Jan. 6, 2010, herein incorporated by reference in their entirety.

### FIELD OF THE INVENTION

This invention relates to a nutritional product for use as a dietary supplement, food, or beverage product as well as an animal feed for suppressing lethality due to general lack of nutrition, enhance longevity under oxidative stress conditions, or prevent and suppress Alzheimer's disease and associated disease states, by use of a mushroom or fungi having a naturally enriched increased vitamin D2 content.

### BACKGROUND OF THE INVENTION

Mushrooms are valuable health food—low in calories, high in vegetable proteins, chitin, iron, zinc, fiber, essential amino acids, vitamins & minerals. Mushrooms also have a long history of use in traditional Chinese medicine. Their legendary effects on promoting good health and vitality and increasing a body's adaptive abilities have been supported by Western medicine as well. They are an excellent source of organic selenium compounds, riboflavin, pantothenic acid, copper, niacin, potassium and phosphorous. Selenium is needed for the proper function of the antioxidant system, which works to reduce the levels of damaging free radicals in the body. Selenium is a necessary cofactor of one of the body's most important internally produced antioxidants, glutathione peroxidase, and also works with vitamin E in numerous vital antioxidant systems throughout the body.

Mushrooms are also a primary source of natural Vitamin D, in the form D2. Most other natural food sources of Vitamin D, in the form Vitamin D3, are of animal, poultry or seafood origin. Also, some foods, such as milk, orange juice and cereals may be fortified with Vitamin D, up to 100 IU per serving.

Vitamin D is a fat-soluble vitamin that is naturally present in very few foods, added to others, and available as a dietary supplement. Vitamin D comes in two forms (D2 and D3) which differ chemically in their side chains. These structural differences alter their binding to the carrier protein vitamin D binding protein (DBP) and their metabolism, but in general the biologic activity of their active metabolites is comparable. It is also produced endogenously when ultraviolet rays from sunlight strike the skin and trigger Vitamin D synthesis. So one must either ingest Vitamin D or sit in the sun and soak up UV rays, so that it may be synthesized endogenously. The risks of sun exposure have gained much attention lately, and the association of sun exposure with pre-cancerous (actinic keratosis) and cancerous (basal cell carcinoma, squamous cell carcinoma and melanoma) skin lesions—caused by loss of the skin's immune function, fine and coarse wrinkling of the skin, freckles, discoloration of the skin, and Elastosis—the destruction of the elastic tissue causing lines and wrinkles is well documented. Thus as people become more sensitive to the dangers of UV exposure, other dietary sources of Vitamin D become increasingly important for maintaining health.

There are two basic types of vitamin D. Ergosterol is the basic building block of vitamin D in plants. Cholesterol is the basic building block of vitamin D in humans. When ultraviolet light from the sun hits the leaf of a plant, ergosterol is converted into ergocalciferol, or vitamin D2. In just the same way, when ultraviolet light hits the cells of our skin, one form of cholesterol found in our skin cells-called 7-dehydrocholesterol-can be converted into cholecalciferol, a form of vitamin D3. The liver and other tissues metabolize vitamin D, whether from the skin or oral ingestion, to 25OHD, the principal circulating form of vitamin D, by the enzyme CYP27B1, the 25OHD-1αhydroxylase. 25OHD is then further metabolized to 1,25(OH)2D principally in the kidney, although other tissues such as epidermal keratinocytes and macrophages contain this enzymatic activity. 1,25(OH)2D is the principal hormonal form of vitamin D, responsible for most of its biologic actions.

Vitamin D is essential for promoting calcium absorption in the gut and maintaining adequate serum calcium and phosphate concentrations to enable normal mineralization of bone and prevent hypocalcemic tetany. It is also needed for bone growth and bone remodeling by osteoblasts and osteoclasts. Without sufficient Vitamin D, bones can become thin, brittle, or misshapen. Vitamin D sufficiency prevents rickets in children and osteomalacia in adults. Together with calcium, Vitamin D also helps protect older adults from osteoporosis.

Vitamin D has many other roles in human health, including modulation of neuromuscular and immune function and reduction of inflammation. Many genes encoding proteins that regulate cell proliferation, differentiation, and apoptosis are modulated in part by Vitamin D. Many laboratory-cultured human cells have Vitamin D receptors and some convert 25(OH)D to 1,25(OH)₂D. It remains to be determined what cells, tissues, and organs in the human body contain either D2, D3, or both vitamin receptors and what additional cells with Vitamin D receptors in the intact human can carry out this conversion from 25(OH)D to 1,25(OH)₂D.

It is an object of the present invention to provide a food product for use in dietary supplements, foods and beverages which is high in nutritional values, particularly Vitamin D2.

It is another object of the invention to provide methods for enhancing the Vitamin D2 content of mushrooms.

It is yet another object of the invention to provide such nutritionally enhanced mushrooms and filamentous fungi without any deleterious effects on the mushrooms appearance, stability, and bioactivity.

It is yet another object of the invention to provide evidence that mushrooms with enhanced Vitamin D2 have different physiologic actions as compared to single nutrient Vitamin D2 and Vitamin D3.

These and other objects of the present invention will become apparent from the description of the invention which follows.

### SUMMARY OF THE INVENTION

This invention creates an improved food product with a naturally enriched vitamin D nutritional profile. The product is obtained by a method comprising the steps of obtaining a mushroom or other fungi the content of Vitamin D or its analogs or derivatives, of which is desired to be increased. The mushroom or fungi is subjected to pulsed UV irradiation. Applicants have discovered the dosage and timing of radiation (pulsing) to provide the highest benefit of increased Vitamin D content, without any negative effects on mushroom appearance, shelf life, or nutrients. These benefits were shown to be stable, even after more than one week in storage.

In yet another embodiment, the Vitamin D enriched mushroom substrate could be used in animal feed or as a nutritional source of Vitamin D. Mushrooms are usually produced by first preparing a substrate, such as corn, oats, rice, millet or rye or various combinations, prepared by soaking the grain in water and sterilizing the substrate before inoculation with mushroom spores or mushroom mycelia. Mycelia are the filamentous hyphae of a mushroom that collect water and nutrients to enable mushrooms to grow. The inoculated substrate is then held to promote colonization of the mycelia, at which point the mycelia-laced grains become “spawn”. This is usually done in individual spawn bags. The substrate provides the nutrients necessary for mycelium growth. The mycelium-impregnated substrate then develops under controlled temperature and moisture conditions, until the hyphae of the mycelium have colonized the substrate. The mycelium enriched product usually is harvested after about four to eight weeks from the beginning of the process, with the contents of the spawn bag possibly processed into dry powdered product. According to the invention, this spent substrate may also be enriched in Vitamin D upon application of pulsed UV irradiation.

As used herein the term “mushroom” or “filamentous fungi” shall be interpreted to include all tissues, cells, organs of the same, including but not limited to mycelium, spores, gills, fruiting body, stipe, pileus, lamellae, basidiospores, basidia, and the like.

As used herein the term “naturally enhanced” with respect to mushrooms and vitamin D, shall mean pulsed UV irradiated mushrooms produced by the methods disclosed herein.

Applicant has found that Vitamin D2 enriched mushrooms produced according to the invention are useful for suppressing lethality due to general lack of nutrition or for enhancing longevity in general and under nutritionally deficient conditions, further elucidating the role of vitamin D₂ in enhancing longevity under stressful conditions and demonstrating another use of the mushrooms of the invention. The vitamin D enriched mushrooms were further shown to increase survival and reduce biologic death under oxidative stress conditions, and to increase survival in biologic models and organisms with Alzheimer's disease. Surprisingly these results were observed in contrast to either vitamin D2 or D3 fed alone, and more surprisingly, the enriched mushrooms were shown to have better survival than non enhanced mushrooms co-adminstered with vitamin D2.

The invention includes pharmaceutical compositions for prevention of, treatment for, and resistance to the effects of oxidative stress, and disease states such as Alzheimer's disease, taupathies and other associated conditions.

### DETAILED DESCRIPTION OF THE FIGURES

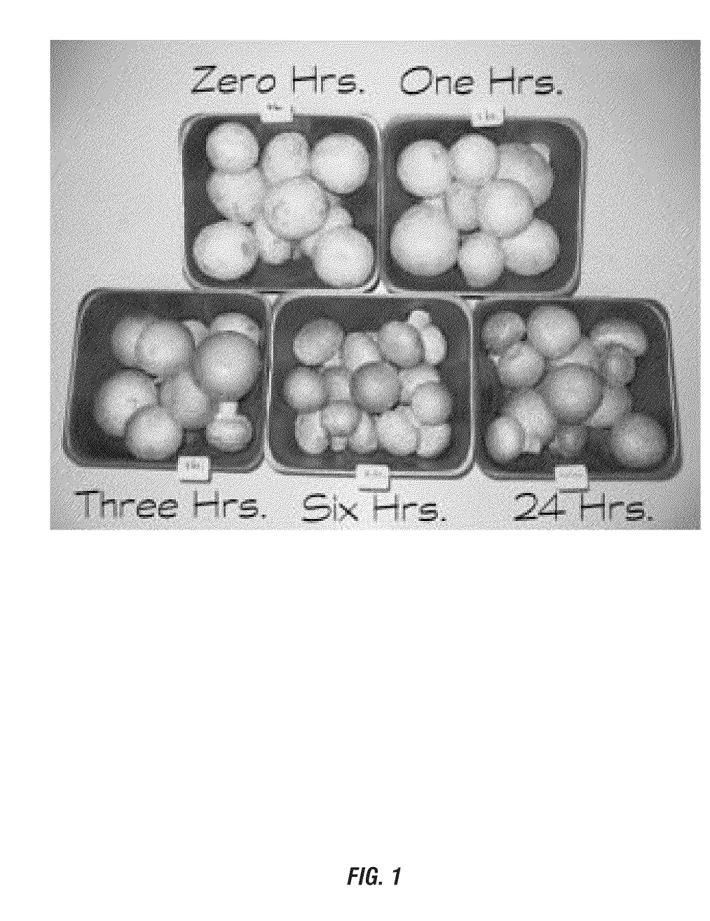

FIG. 1 is a photograph of UV treated mushrooms by the methods of Feeney et al.

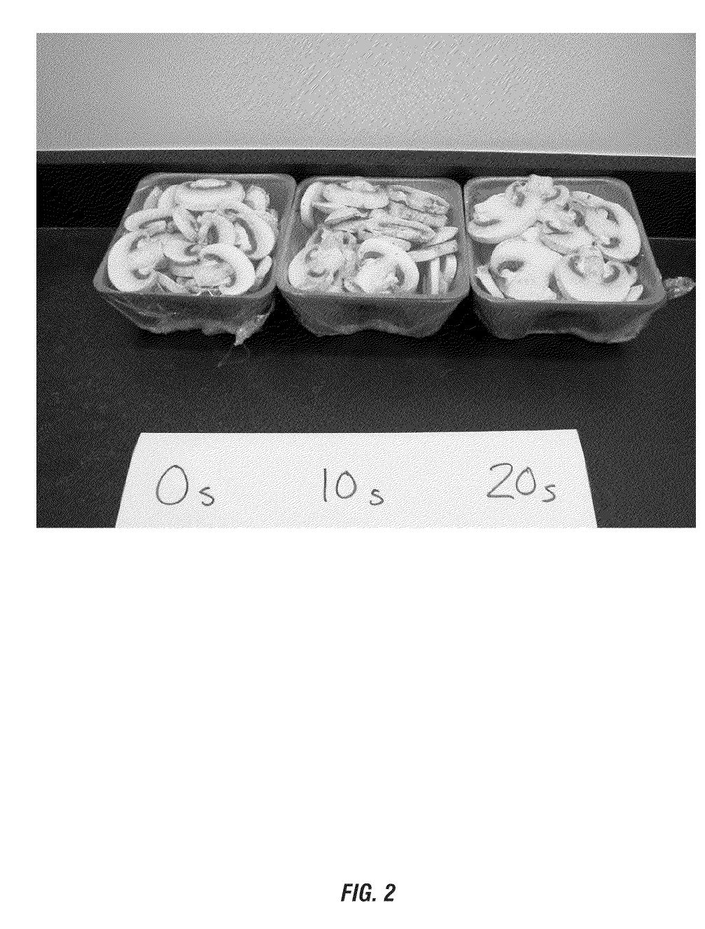

FIG. 2 is a photograph of pulsed UV treated mushrooms according to the invention.

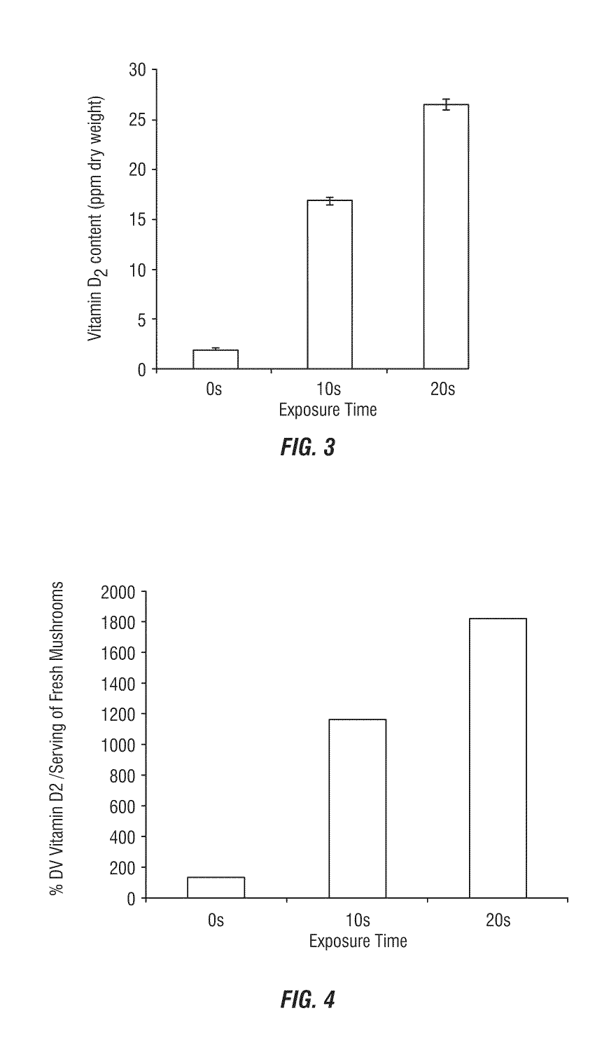

FIG. 3 is a graph depicting the Vitamin D₂ content of fresh sliced mushrooms after exposure to pulsed UV-light at 0, 10 and 20 seconds (C-type lamp).

FIG. 4 is a graph depicting the Percent DV of vitamin D₂ in one serving of fresh sliced mushrooms after exposure to pulsed UV-light at 0, 10 and 20 seconds (C-type lamp).

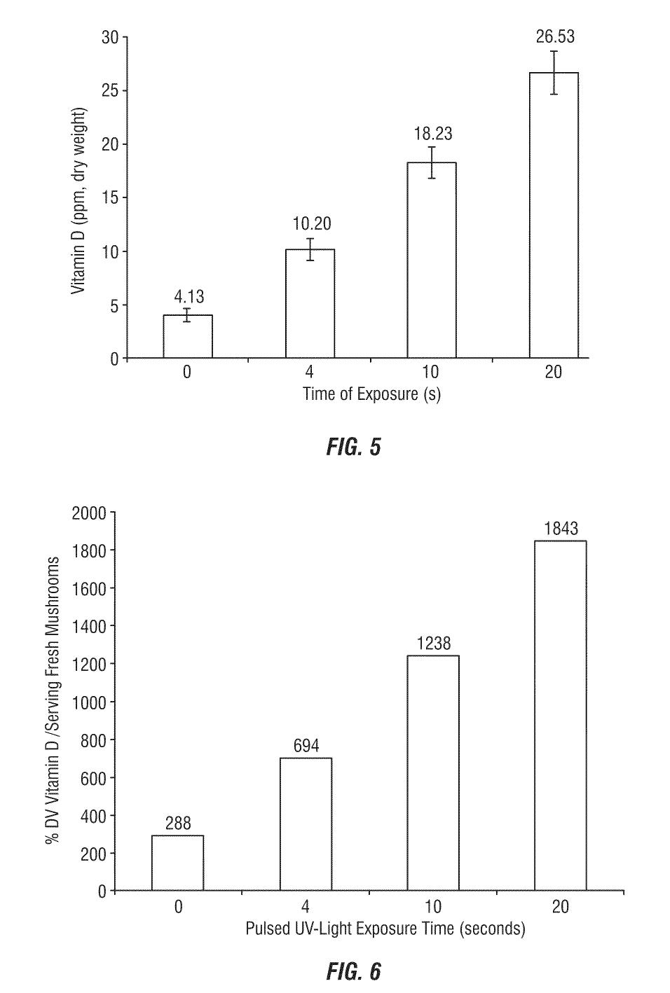

FIG. 5 is a graph depicting the Vitamin D₂ content of fresh sliced mushrooms after exposure to pulsed UV light at 0, 4, 10 and 20 seconds (C-type lamp).

FIG. 6 is a graph depicting the Percent DV of Vitamin D₂ in one serving of fresh sliced mushrooms after exposure to pulsed UV-light (C-type lamp).

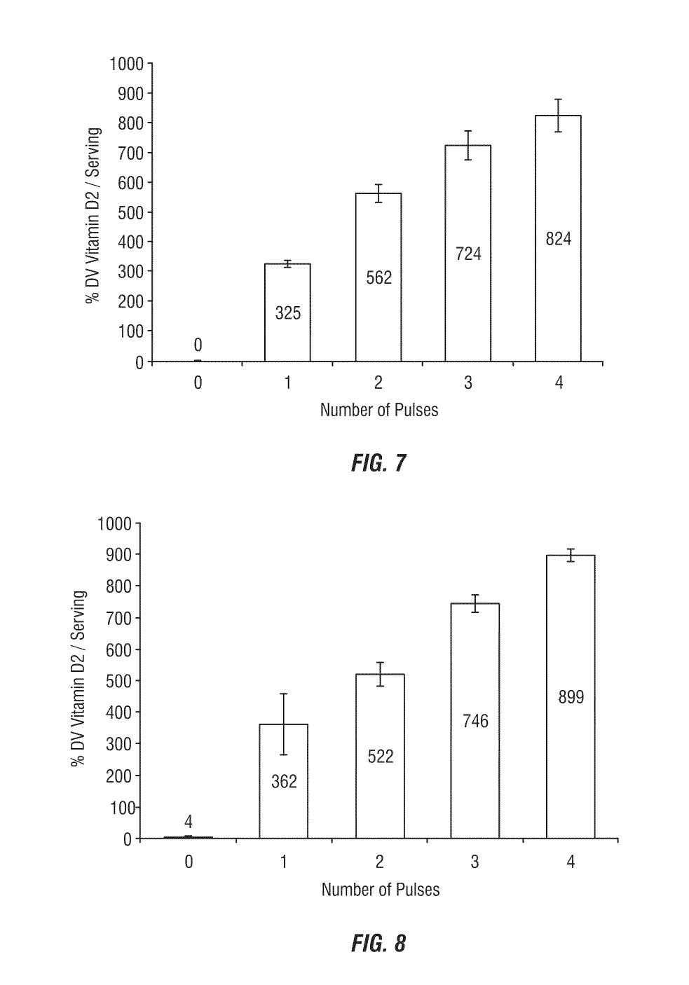

FIG. 7 is a graph showing the Percent DV Vitamin D₂ in one serving (84 g) of white button mushrooms (Agaricus bisporus) after pulsed UV light exposure (B-type lamp). Error bars represent standard deviation of the three replications.

FIG. 8 is a graph depicting the percent DV Vitamin D₂ in one serving (84 g) of brown button mushrooms (Agaricus bisporus) after pulsed UV light exposure (B-type lamp). Error bars represent standard deviation of the three replications.

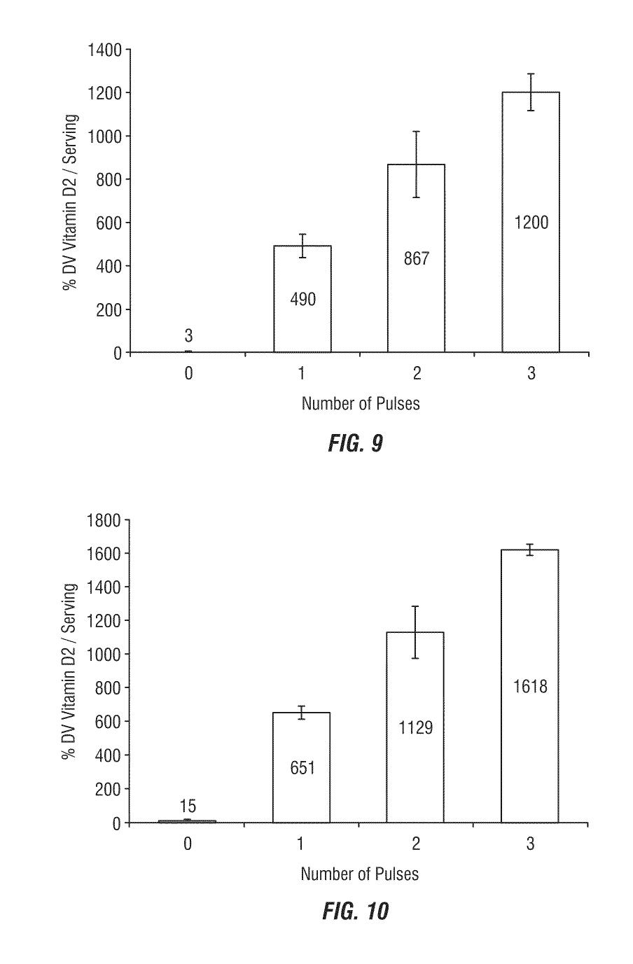

FIG. 9 is a graph depicting the percent DV Vitamin D₂ in one serving (84 g) of shiitake mushrooms (Lentinula edodes) after pulsed UV light exposure (B-type lamp). Error bars represent standard deviation of the two replications.

FIG. 10 is a graph showing the percent DV Vitamin D₂ in one serving (84 g) of oyster mushrooms (Pleurotus ostreatus) after pulsed UV light exposure (B-type lamp). Error bars represent standard deviation of the two replications.

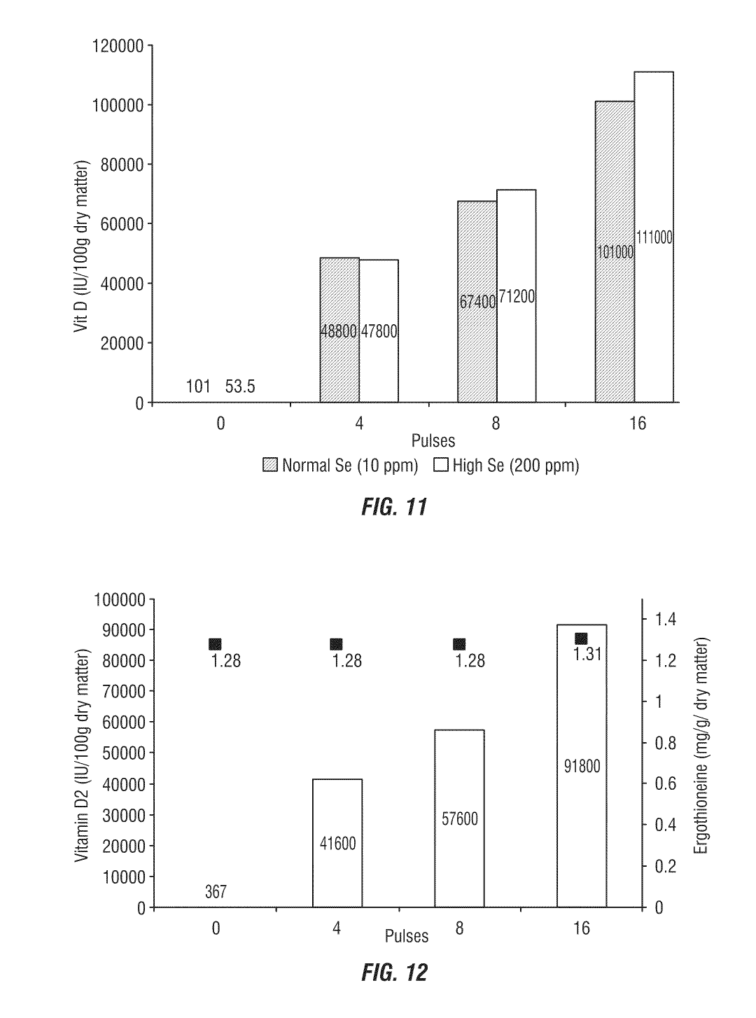

FIG. 11 is a graph showing the vitamin D₂ content of pulsed UV treated (B-type lamp) selenium enriched and normal air-dried Agaricus bisporus mushroom powder. Samples were treated at a distance of 3.2 cm.

FIG. 12 is a graph showing the Vitamin D₂ and ergothioneine (black squares) contents of pulsed UV treated (B-type lamp) king oyster mushroom powder. Samples were treated at a distance of 3.2 cm.

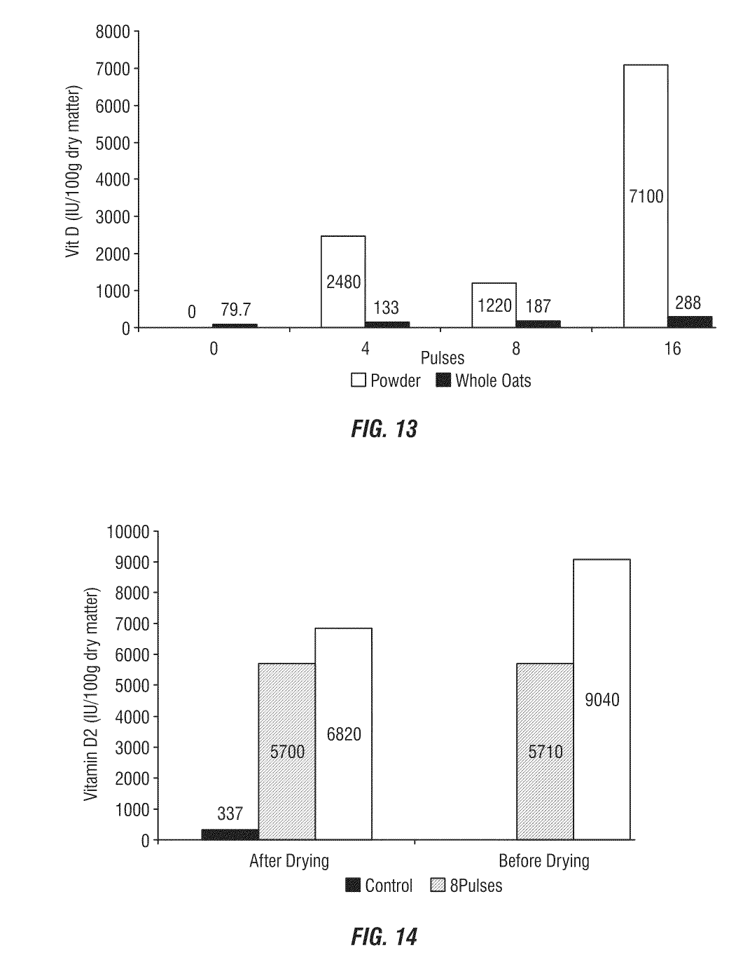

FIG. 13 is a graph depicting the Vitamin D₂ content of pulsed UV treated (B-type lamp) king oyster mycelium grown on oat substrate. Samples were treated at a distance of 3.2 cm in both whole oat and ground powder form.

FIG. 14 is a graph showing the Vitamin D₂ content of pulsed UV treated (B-type lamp) spent king oyster substrate. Samples were treated wet and dry at a distance of 3.2 cm.

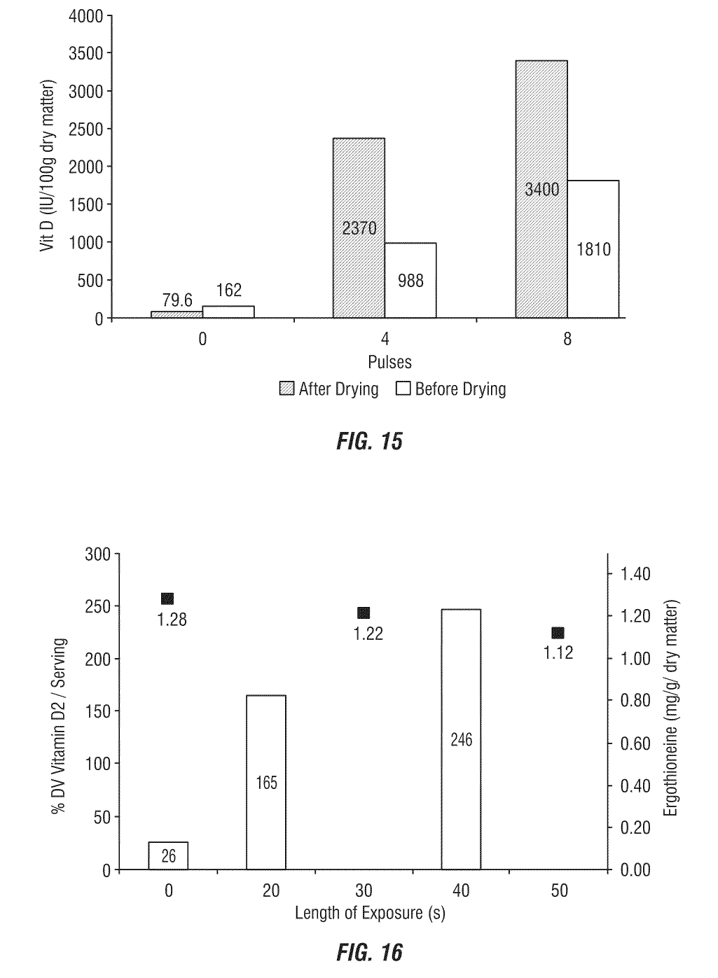

FIG. 15 is a graph depicting the Vitamin D₂ content of pulsed UV treated (B-type lamp) spent maitake substrate. Samples were treated at a distance of 3.2 cm before and after air-drying.

FIG. 16 is a graph showing the Vitamin D₂ and ergothioneine (black squares) contents of pulsed UV treated (C-type lamp) Agaricus bisporus. Samples were treated at a distance of 8 cm.

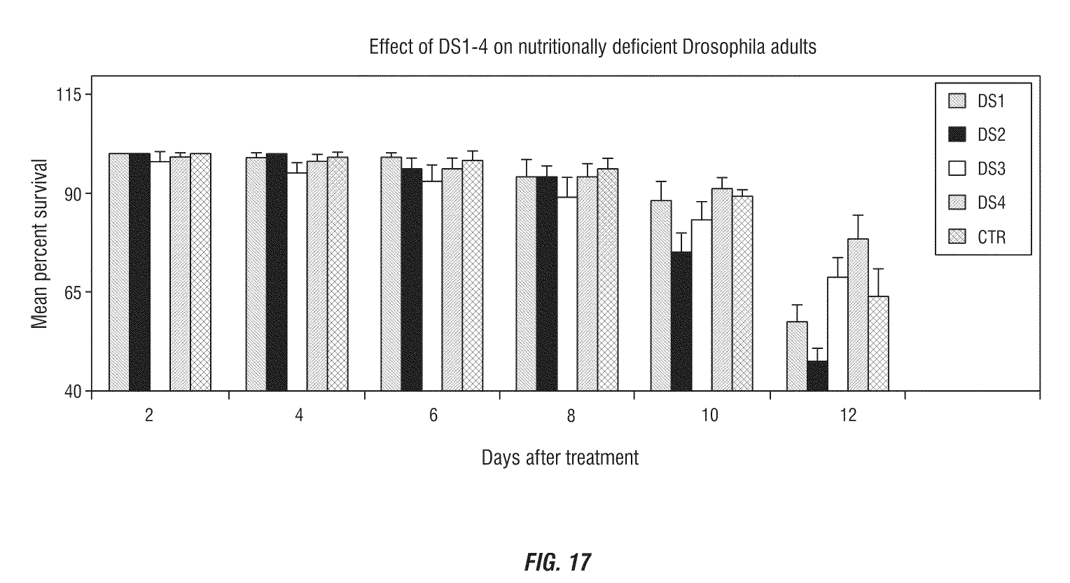

FIG. 17 is a graph depicting means percent survival of Drosophila treated with Vitamin D enriched Agaricas blazei per days of treatment. For control, Agaricus blazei non enriched and the food base alone were used.

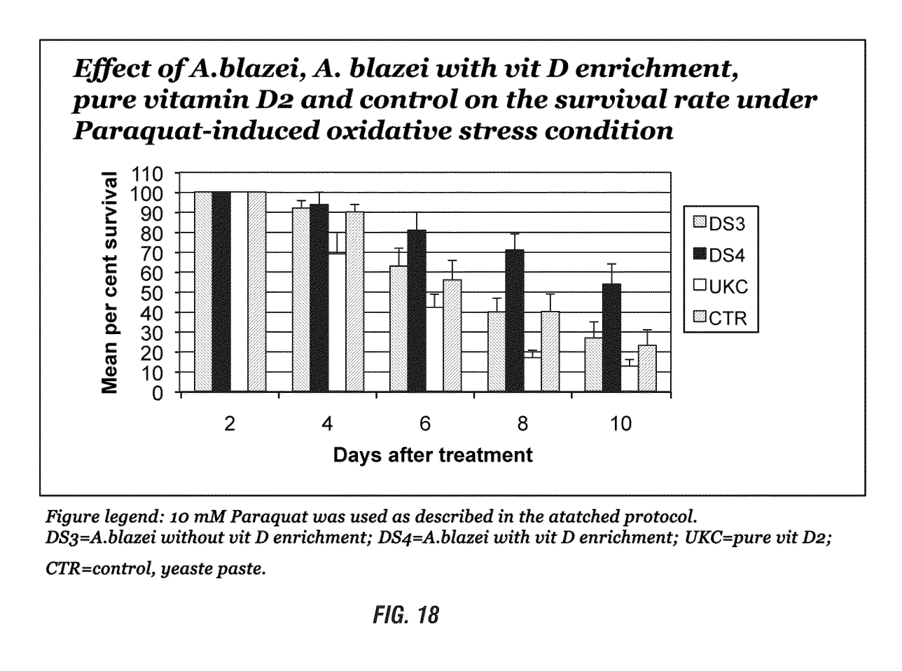

FIG. 18 is a graph showing the percent survival of Drosophila treated with Vitamin D enriched Agaricas blazei per days of treatment.

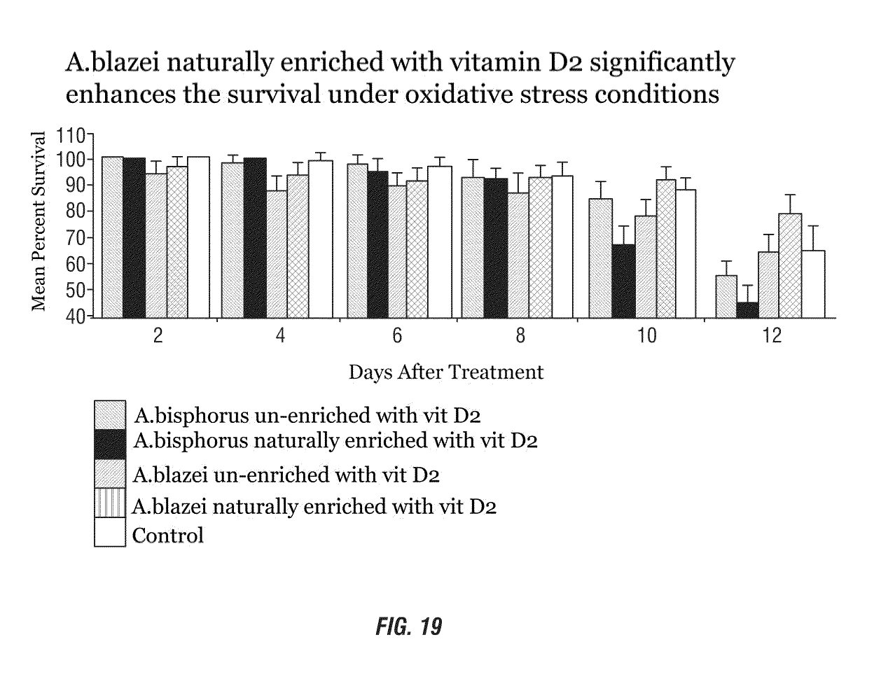

FIG. 19 is a graph showing Drosophila survival under oxidative stress with A. blazei naturally enriched with vitamin D2. The results show that the enriched mushrooms significantly enhance survival.

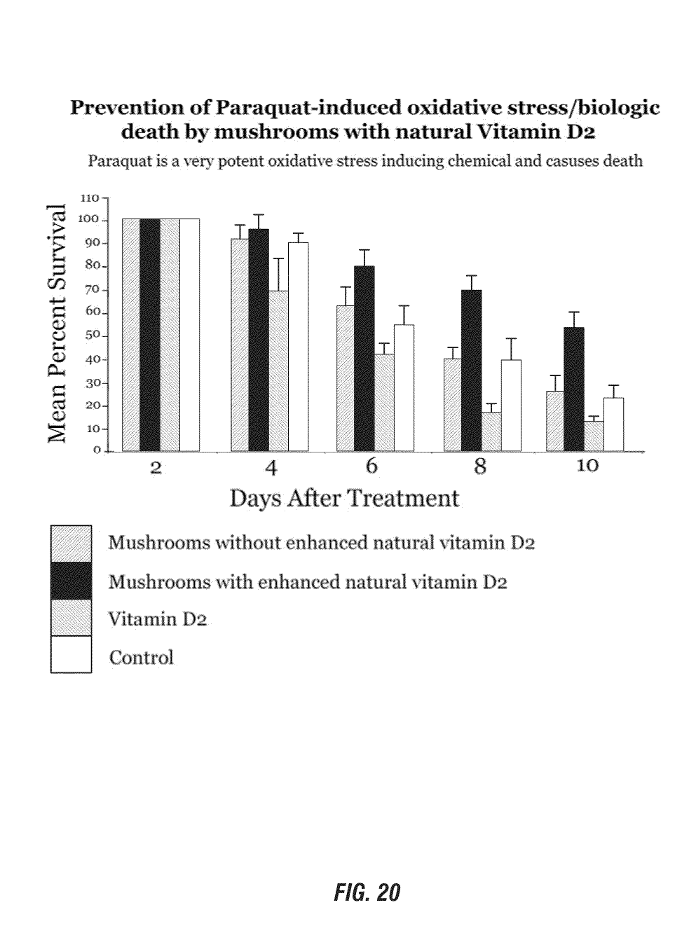

FIG. 20 is a graph showing Drosophila survival and prevention of death under Paraquat induced oxidative stress with A. blazei naturally enriched with vitamin D2. The results show that the enriched mushrooms significantly enhance survival.

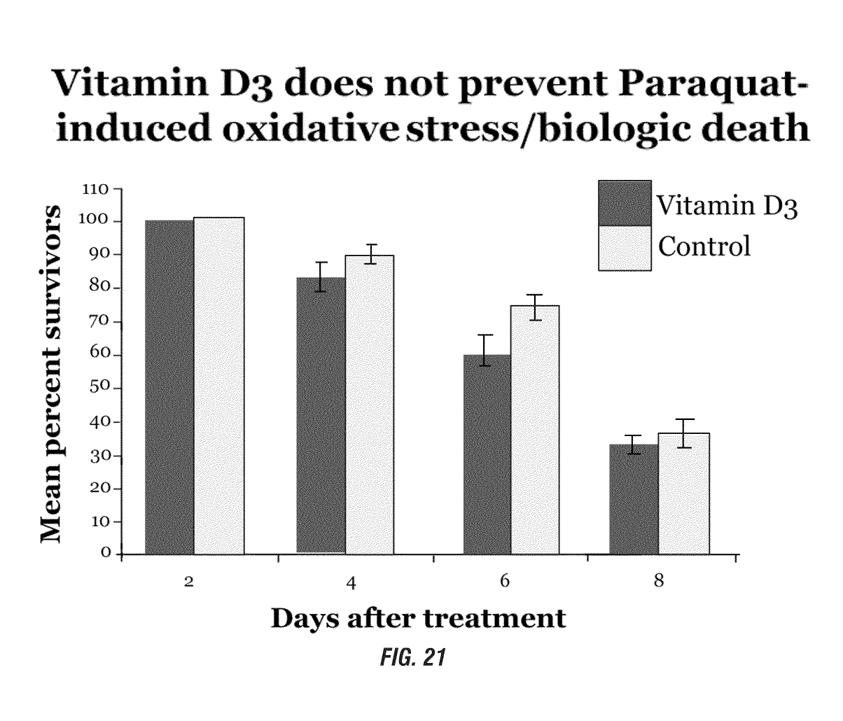

FIG. 21 is a graph showing Paraquat induced oxidative stress survival with vitamin D3 treatment. The results indicate that vitamin D3 does not prevent biologic death.

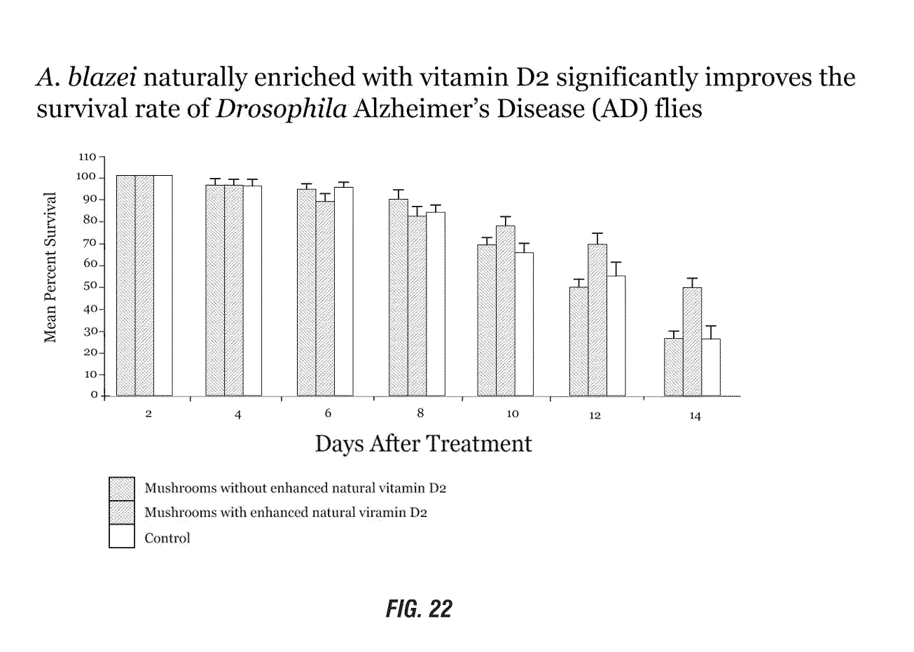

FIG. 22 is a graph showing A. blazei enriched vitamin D2 and the survival rate of Drosophila Alzheimer's disease flies. The results show that the enriched mushrooms increase survival in an Alzheimer's disease model.

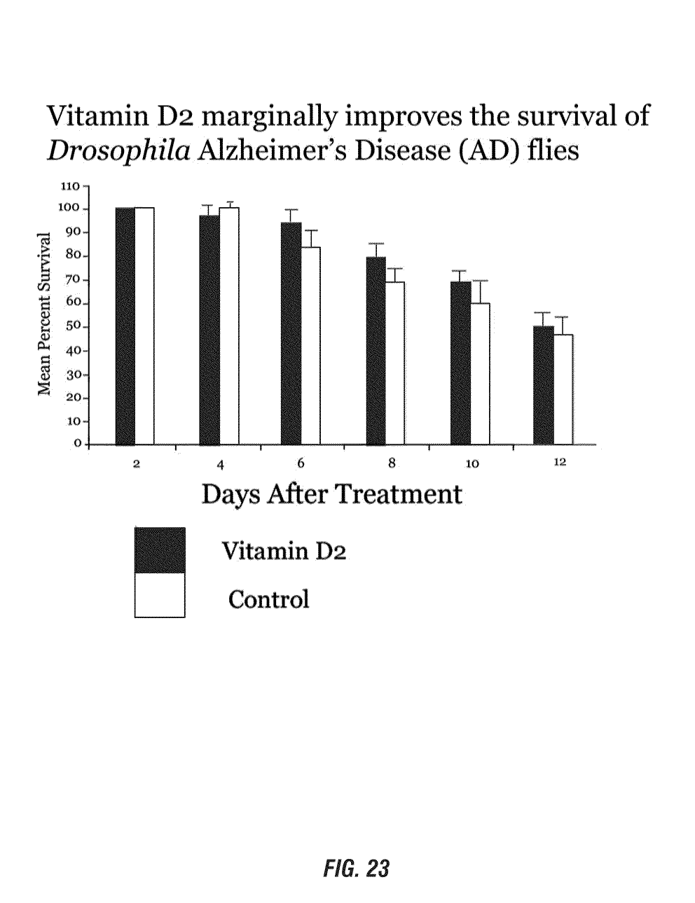

FIG. 23 is a graph showing that vitamin D2 alone only marginally increases survival of Drosophila Alzheimer's disease flies.

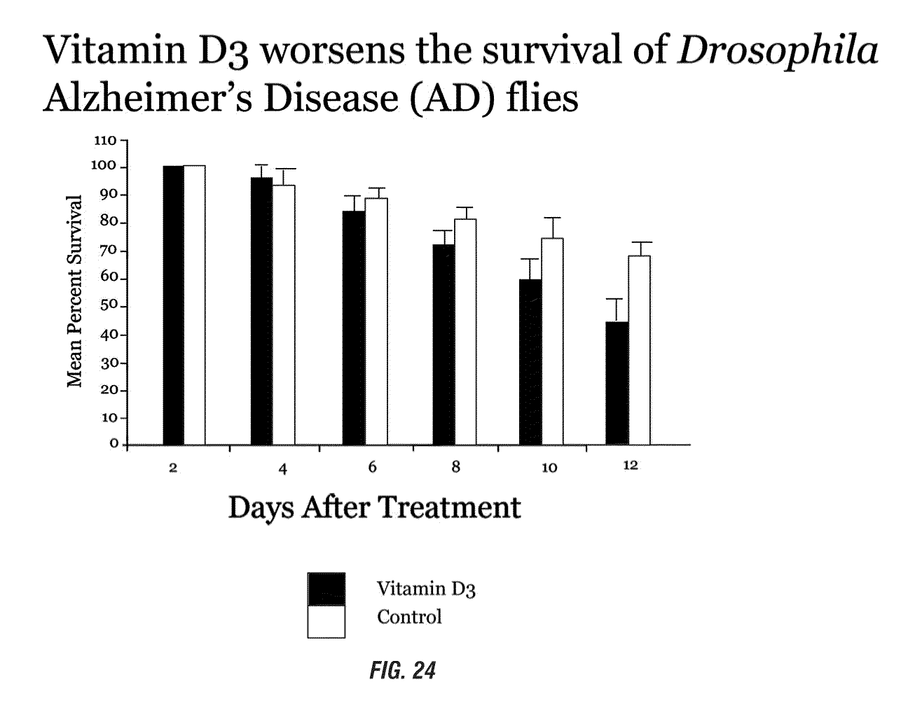

FIG. 24 is a graph showing the effects of vitamin D3 on the survival of Drosophila Alzheimer's disease flies. The results show that vitamin D3 actually decreases survival of the flies.

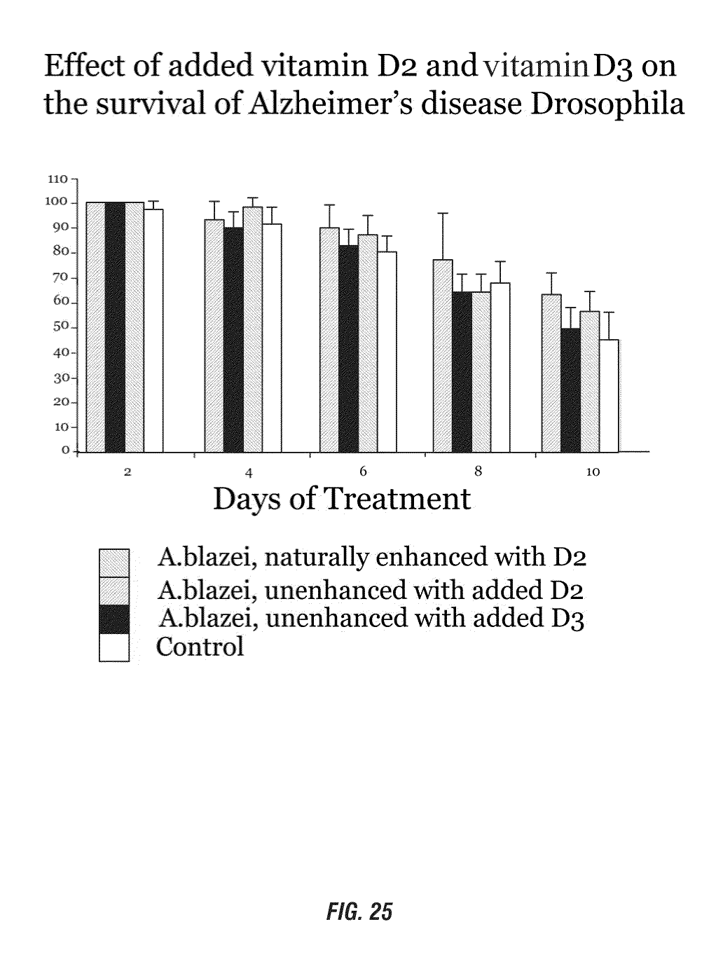

FIG. 25 is a graph showing the effects of added vitamin D2 and D3 compared with the enriched mushrooms on the survival of Drosophila Alzheimer's disease flies. The results show that the enhanced mushrooms have the greatest increase in survival.

### DETAILED DESCRIPTION OF THE INVENTION

Previous research (Feeney, 2006) determined that exposing mushrooms to constant ultraviolet light can produce Vitamin D₂ by converting the naturally-occurring ergosterol to Vitamin D₂. However, there were concerns about compliance with nutrition labeling regulations throughout retail distribution, deleterious effects on appearance, and tissue browning. Another significant disadvantage was the increased length of exposure time required by conventional sources of UV light, which were impractical in a packinghouse environment. Thus constant UV radiation at sufficient time and strength caused deleterious effects on the appearance of mushrooms and at best, achieved an increase of 100% of the % DV/per serving of Vitamin D but with a host of regulatory, and commercial processing concerns.

Chikthimmah and Beelman (2006) recently tested pulsed UV-light treatments at very high levels for long period of time (30 seconds or more) to reduce bacterial populations in fresh mushrooms. In this paper, they speculated that Vitamin D₂ content in mushrooms could be rapidly increased using pulsed UV-light. The conclusion was, however that such exposure caused discoloration and deleterious effects on the appearance of mushrooms, particularly white mushrooms. Such browning of mushrooms would make them commercially undesirable. Bacterial populations are responsible for the browning and degradation of mushrooms, which has a dramatic and negative effect on their appeal to customers.

According to the invention, applicant herein demonstrates that pulsed UV light at lower ranges and for very brief periods was shown to have dramatic increases in the Vitamin D levels present in such mushrooms, with increases by as much as 800 times the % DV (percent daily value) of Vitamin D content, per serving with no deleterious effects on the morphology or appearance of the mushroom. This dramatic increase in Vitamin D content in light of earlier studies which had demonstrated less Vitamin D conversion after much longer periods of UV exposure is quite surprising.

Pulsed UV-light treatments to increase Vitamin D₂ content in mushrooms were conducted with a laboratory scale, pulsed light sterilization system (SteriPulse®-XL 3000, Xenon Corporation, Woburn, Mass.) that is present in the Department of Agricultural Biological Engineering at Penn State. While applicants postulate that it is the UVB component of the Xenon pulsed light system that is responsible for the effects of the invention, it should be noted that the system uses pulsed light which includes the entire spectrum of light and may also include other components that contribute to the effects demonstrated herein and which are intended to be within the scope of the invention.

According to the invention, pulses of UV radiation of approximately 1-10 J/cm² per pulse, preferably 3-8 J/cm² and most preferably 5-6 J/cm² is used. Voltages may also vary based upon safety concerns but should generally be in the range of 1 to 10 or even up to 100 or 10,000 volts as safety mandates. The pulses should generally be in a range of 1-50 pulses per second more preferably 1-30 pulses per second and most preferably 1-10 pulses per second for a range of treatment post harvest of 0 to 60 seconds.

Any type of mushroom, mushroom part, component, fungi or even used substrate for cultivating mushrooms, with ergosterol present may be used. This includes all filamentous fungi where ergosterol has been shown to be present and includes the use of tissues such as the mycelia, spores or vegetative cells. This includes, but is not limited to, for example, Coprinus, Agrocybe, Hypholoma, Hypsizygus, Pholiota, Pleurotus, Stropharia, Ganoderma, Grifola, Trametes, Hericium, Tramella, Psilocybe, Agaricus, Phytophthora achlya, Flammulina, Melanoleuca, Agrocybe, Morchella, Mastigomycotina, Auricularia, Gymnopilus, Mycena, Boletus, Gyromitra, Pholiota, Calvatia, Kuegneromyces, Phylacteria, Cantharellus, Lactarius, Pleurotus, Clitocybe, Lentinula (Lentinus), Stropharia, Coprinus, Lepiota, Tuber, Tremella, Drosophia, Leucocoprinus, Tricholoma, Dryphila, Marasmius, and Volvariella.

Non-limiting examples of other fungal genera, including fermentable fungi, include: Alternaria, Endothia, Neurospora, Aspergillus, Fusarium, Penicillium, Blakeslea, Monascus, Rhizopus, Cephalosporium, Mucor, and Trichoderma

In yet another embodiment, the spent mushroom substrate upon which mushrooms are cultivated, was enriched in Vitamin D using pulsed UV light according to the invention. Such spent substrate could then be used as nutritional feed supplements and the like for mammalian animals, fish, shrimp, chickens, and other similar edible species.

The inventors used 5.61 J/cm² per pulse on the strobe surface for an input voltage of 3800V and with 3 pulses per second. Sliced mushrooms (Agaricus bisporus, white strain) were placed in the pulsed UV-light sterilization chamber and treated with pulsed light for up to a 20-second treatment at a distance of 17 cm from the UV lamp or 11.2 cm from the window. Control samples did not undergo any pulsed UV treatment. Treated mushrooms were freeze-dried and then sent to a selected commercial laboratory for Vitamin D₂ analysis. In this study, a pulsed UV system was also evaluated for effects on the appearance of fresh mushroom slices during a shelf life study.

Results of the experiments demonstrated that pulsed UV-light was very effective in rapidly converting ergosterol to Vitamin D₂. Control mushrooms contained 2 ppm d.w. Vitamin D₂, while 10 and 20 seconds of exposure to pulsed UV-light resulted in 17 and 26 ppm Vitamin D₂, respectively (FIG. 1). This increase was equivalent to over 1800% DV Vitamin D in one serving of fresh mushrooms after a 20 second exposure to pulsed UV (FIG. 2). The mushrooms treated for 20 seconds also showed no noticeable difference in appearance initially as well as after 10 days of storage at 3° C. compared to the untreated control.

These results compared favorably to the previous pilot study (Feeney, 2006) where mushrooms were exposed to 5 minutes of conventional UV-light exposure. In that study, the mushrooms contained 14 ppm Vitamin D₂, but they were also significantly discolored. Hence, the pulsed UV method shows considerable promise as a rapid means to enhance Vitamin D₂ levels in fresh mushrooms, theoretically reducing required exposure times from minutes to seconds. Pulsed UV-light exposure did not result in any negative effects on mushroom quality.

Another experiment revealed that pulsed UV-light could rapidly convert ergosterol present in dried oyster mushroom powder to Vitamin D₂ (Table 1). These findings indicate that this technology could be used to enrich other mushroom products with Vitamin D₂.

TABLE 1
Vitamin D₂ generation in dried oyster mushroom
powder exposed to pulsed UV-light (C-type lamp).

| Time of Exposure(s) | Vitamin D₂ (PPM) |
|---|---|
| 0 | 8.5 |
| 8 | 15.18 |
| 16 | 24.24 |

The present invention relates to methods for obtaining a nutritionally enhanced food product using pulsed UV radiation to increase Vitamin D and/or its derivatives in filamentous fungi. The solid substrate can be any part of the mushroom or mold, including the mycelia, spores etc, so long as ergosterol is present in at least part of the tissue or cells.

In the present invention, the filamentous fungi product is subjected to pulsed UV irradiation after harvest, being irradiated with UV light for a time sufficient to enhance the Vitamin D content thereof. By utilizing UV irradiation, the food product has a substantially increased level of Vitamin D. Preferably, the food product is irradiated with UV radiation, specifically Ultraviolet-B (UV-B), a section of the UV spectrum, with wavelengths between about 280 and 320 nm, or Ultraviolet-C (UV-C), with wavelengths between about 200 and 280 nm. In a more preferred embodiment the UV radiation is pulsed. It is believed that the additional Vitamin D is obtained through the conversion of ergosterol due to the UV irradiation. The time may be the same or increased when the irradiation occurs during the growing process, or post harvest though the UV irradiation can occur during both periods.

Applicant has further demonstrated that Vitamin D enriched mushrooms increase longevity in Drosophila kept under nutritionally deficient diet and thus represent a novel use of the mushrooms of the invention as well as further elucidating the role of Vitamin D, particularly Vitamin D₂ in aging. According to the invention, applicant has also shown that the naturally enriched vitamin D mushrooms of the invention increase survival and decrease biologic death in conditions associated with oxidative stress and also in disease states such as Alzheimer's disease. Thus the invention includes supplements, pharmaceutical compositions, and like employing the mushrooms or components thereof and a carrier. Quite surprisingly, applicants have demonstrated that administration of Vitamin D2 or Vitamin D3 alone does not have the same effects as the enriched mushrooms of the invention.

In one embodiment, in addition to extracts, fractions thereof or compounds thereof or compounds isolated from the enriched mushrooms of the invention, the compositions of the present invention may include a pharmaceutically acceptable carrier.

In order to facilitate administration, the extracts, fractions thereof, compounds derived from or the enriched mushrooms of the invention themselves may be mixed with any of a variety of pharmaceutically acceptable carriers for administration. “Pharmaceutically acceptable” as used herein means that the extract, fraction thereof, or compound thereof or composition is suitable for administration to a subject to achieve the treatments described herein, without unduly deleterious side effects in light of the severity of the disease and necessity of the treatment. The carrier may be a solid or a liquid, or both, and is preferably formulated with the compound as a unit-dose formulation, for example, a tablet, which may contain from 0.5% to 95% by weight of the active compound. One or more of each of the enriched mushroom extracts, fractions thereof or compounds thereof of the present invention may be incorporated in the formulations of the invention, which may be prepared by any of the well known techniques of pharmacy consisting essentially of admixing the components, optionally including one or more accessory ingredients. In one embodiment, the extracts, fractions, and compounds of this invention may be administered in conjunction with other medicaments known to those of skill in the art.

Other compatible pharmaceutical additives and actives may be included in the pharmaceutically acceptable carrier for use in the compositions of the present invention.

One embodiment includes administering a composition for the treatment of oxidative stress or disease states or conditions associated therewith such as Alzheimer's, including an extract, fraction thereof or compound thereof and a carrier. As used herein, a subject may be a human, non-human primate, cow, horse, pig, sheep, goat, dog, cat, rodent, fish, shrimp, chicken, and the like.

Dose ranges can be adjusted as necessary for the treatment of individual patients and according to the specific condition treated. Any of a number of suitable pharmaceutical formulations may be utilized as a vehicle for the administration of the compositions of the present invention and maybe a variety of administration routes are available. The particular mode selected will depend of course, upon the particular formulation selected, the severity of the disease, disorder, or condition being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, nasal, transdermal or parenteral routes and the like. Accordingly, the formulations of the invention include those suitable for oral, rectal, topical, buccal, parenteral (e.g., subcutaneous, intramuscular, intradermal, inhalational or intravenous) and transdermal administration, although the most suitable route in any given case will depend on the nature and severity of the condition being treated and on the nature of the particular active product used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above).

In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations of the present invention suitable for parenteral administration conveniently comprise sterile aqueous preparations of the active compound, which preparations are preferably isotonic with the blood of the intended recipient. These preparations may be administered by means of subcutaneous, intravenous, intramuscular, inhalational or intradermal injection. Such preparations may conveniently be prepared by admixing the compound with water or a glycine buffer and rendering the resulting solution sterile and isotonic with the blood. Alternately, the extracts, fractions thereof or compounds thereof can be added to a parenteral lipid solution.

Formulations of the inventive mixtures are particularly suitable for topical application to the skin and preferably take the form of an ointment, cream, lotion, paste, gel, spray, aerosol, or oil. Carriers which may be used include vaseline, lanoline, polyethylene glycols, alcohols, transdermal enhancers, and combinations of two or more thereof.

Formulations suitable for transdermal administration may also be presented as medicated bandages or discrete patches adapted to remain in intimate contact with the epidermis of the recipient for a prolonged period of time. Formulations suitable for transdermal administration may also be delivered by iontophoresis (passage of a small electric current to “inject” electrically charged ions into the skin) through the skin. For this, the dosage form typically takes the form of an optionally buffered aqueous solution of the active compound. Suitable formulations comprise citrate or bis/tris buffer (pH 6) or ethanol/water and contain from 0.01 to 0.2M active ingredient.

Mammals may be treated using the methods of the present invention and are typically human subjects although the methods of the present invention may be useful for veterinary purposes with other subjects, particularly mammalian subjects including, but not limited to, horses, cows, dogs, rabbits, fowl, sheep, and the like. As noted above, the present invention provides pharmaceutical formulations comprising extracts, fractions thereof or compounds thereof or combinations thereof of the present invention, or pharmaceutically acceptable salts thereof, in pharmaceutically acceptable carriers for any suitable route of administration, including but not limited to oral, rectal, topical, buccal, parenteral, intramuscular, intradermal, intravenous, and transdermal administration.

The therapeutically effective dosage of any specific compound will vary somewhat from compound to compound, patient to patient, and will depend upon the condition of the patient and the route of delivery. As a general proposition, a dosage from about 0.01 to about 50 mg/kg will have therapeutic efficacy, with still higher dosages potentially being employed for oral and/or aerosol administration. Toxicity concerns at the higher level may restrict intravenous dosages to a lower level such as up to about 10 mg/kg, all weights being calculated based upon the weight or volume of the enriched mushrooms, fractions thereof or compounds thereof of the present invention, including the cases where a salt is employed.

The present invention also provides medical foods comprising the enriched mushrooms of the invention including extracts, fractions thereof or compounds thereof or any combination thereof, the medical food being compounded for the amelioration of a disease, disorder or condition associated with or caused by oxidative stress.

This invention can be better understood by reference to the following non-limiting examples. It will be appreciated by those skilled in the art that other embodiments of the invention may be practiced without departing from the spirit and the scope of the invention as herein disclosed and claimed.

### REFERENCES

1. Chikthimmah, N., Beelman, R. B. Microbial spoilage of fresh mushrooms. In: Microbiology of Fruits and Vegetables, Sapers et al, Ed. Pgs 135-158. Taylor and Francis (2006).
2. Feeney, M. J., Optimizing Vitamin D₂ in mushrooms; Pilot study to expose mushrooms to ultraviolet light. Mushroom New 54 (5):2-24 (2006).
3. Johnson, G. H., Increasing the D-mand for mushrooms with Vitamin D. Mushroom News 54 (8):20-23 (2006).

### Example 1

Fresh mushrooms were obtained from Modern Mushroom Farm (Avondale, Pa.) and the Penn State MTDF. All mushrooms were protected from extraneous light exposure throughout the experiments.

A Steripulse®-XL 3000 (Xenon Corporation, Wilmington, Mass.) was used for Pulsed UV light exposure. A B-type lamp was used. The system generated 505 Joules per pulse. At 3.2 cm from the window or 9 cm from the lamp, the broadband energy was 0.873 J/cm² per pulse. The system generates 3 pulses per second. All previous experiments were conducted using a Xenon C-type lamp.

Brown and white button mushrooms were sliced to expose gill tissue. They were randomly placed in 150 g lots into polystyrene containers. Oyster and Shiitake mushrooms were divided into 150 g lots and were arranged in the system so that there was a single layer of mushrooms. All samples were placed in the Pulsed UV system at a distance of 3.2 cm from the quartz window.

Brown and white button mushrooms were exposed for 0, 1, 2, 3, and 4 pulses. All treatments were repeated three times. Oyster and Shiitake mushrooms were exposed for 0, 1, 2, and 3 pulses. All treatments were repeated twice.

Mushroom powders from air-dried Agaricus bisporus with and without selenium enrichment grown at the Penn State MTDF using the methods of Werner and Beelman (2002), were treated in 5 g lots in uncovered Petri plates at a distance of 3.2 cm from the quartz window. King Oyster mushrooms (obtained from Golden Gourmet Mushrooms, San Marcos, Calif.) were air-dried and treated at the same distance. The powders were treated at 0, 4, 8, and 16 pulses.

Spent mushroom substrates (Maitake and King Oyster) obtained from Golden Gourmet Mushrooms were treated either before or after air drying. Dry samples were treated in 5 g lots and wet samples were treated in 20 g lots. The Maitake substrate was treated at 0, 4, and 8 pulses. King Oyster substrates were treated at 0, 8 and 16 pulses.

Commercially dried King Oyster mycelial biomass grown on sterile organic oats at Golden Gourmet Mushrooms (Mushroom Matrix) were treated at 0, 4, 8, and 16 pulses before and after being ground into powder form.

The King Oyster mushroom powder was also evaluated for ergothioneine content. Ergothioneine content was determined by the method of Dubost et al (2006). Ergothioneine levels are reported as mg/g dry weight.

Mushroom samples were freeze-dried directly following treatment and ground into powder. All other samples were air-dried and ground into powders. The powders were sent to Medallion Labs (Minneapolis, Minn.) for Vitamin D₂ analysis.

Vitamin D₂ values of fresh mushrooms are presented based on the % DV (Adequate Intake of 400 IU) in a serving (84 g) of fresh mushrooms. Vitamin D₂ values for powders and substrates are presented as IU/100 g dry weight.

### Results and Discussion

After exposure to increasing amounts of pulsed UV light there was an increase in Vitamin D₂ content of every mushroom product tested. With each additional pulse the mushrooms were exposed to increasing amounts of irradiation and thus more energy was available for Vitamin D₂ synthesis from ergosterol.

Fresh sliced white button mushrooms showed an increase from an initial Vitamin D₂ level of 0% DV/serving to 325% DV/serving after just one pulse (FIG. 5). After 4 pulses the level of Vitamin D₂ increased to 824% DV/serving.

Fresh sliced brown button mushrooms (FIG. 6) Vitamin D₂ went from an initial level of 4% DV/serving at 0 pulses to 362% DV/serving after one pulse. The level increased to 899% DV/serving after 4 pulses.

After Pulsed UV treatment fresh Shiitake mushrooms (FIG. 7) showed an increase in Vitamin D₂ content from an initial level of 3% DV/serving at 0 pulses to 490% DV/serving after one pulse. The Vitamin D₂ content after 3 pulses was 1200% DV/serving.

Fresh Oyster mushrooms contained an initial level of Vitamin D₂ of 15% DV/serving at 0 pulses to a level of 1618% DV/serving after 3 pulses (FIG. 8).

The Oyster and Shiitake showed higher amounts of Vitamin D₂ content after Pulsed UV light exposure than the brown and white button mushrooms. This is most likely due to the thickness of the layer of mushrooms in the system. The brown and white button mushrooms were placed in polystyrene containers to simulate a package of sliced mushrooms being treated. The Oyster and Shiitake mushrooms were treated as whole mushrooms since their geometry did not permit for even distribution when packed together. The single layer of the Oyster and Shiitake mushrooms was similar to how these mushrooms would be treated if the Pulsed UV system were placed over a line where the mushrooms were being transported on a conveyor belt in a single layer. An additional study would be needed to directly compare the Vitamin D₂ content of the Agaricus mushrooms to the Oyster and Shiitake mushrooms.

This study demonstrates that after a very short exposure time of about 1 sec (system generates 3 pulses per second) the Vitamin D₂ content of these mushroom varieties can be increased from very little to upwards of 800% DV/serving. Previous studies using continuous UV light has been shown to take at least 5 minutes of exposure to obtain similar values (Feeney, 2006).

This study also showed that increasing the Vitamin D₂ contents of several mushroom products such as powders and substrates is possible. This material could be used as food ingredients or for animal feed to create value added products.

FIG. 9 shows that the Vitamin D₂ content of selenium enriched (200 ppm) and control (10 ppm) mushroom powder (Agaricus bisporus) were increased in a similar manner from around 100 IU at 0 pulses to over 100,000 IU per 100 g with a treatment of 16 pulses.

The Vitamin D₂ content of air-dried King Oyster powder was increased from 367 IU at 0 pulses to 91800 IU per 100 g after 16 pulses. The ergothioneine content of the dried products remained constant around 1.3 mg/g for all treatments (FIG. 10) indicating that pulsed UV treatment had no effect on ergothioneine levels.

Mushroom mycelial biomass grown on sterile organic oats showed similar increases in Vitamin D₂ with increasing exposure although levels were not as high as with pure fruiting body material. Vitamin D₂ dried King Oyster mycelial biomass increased significantly when ground from 0 to 7100 IU, however when exposed before grinding the level only rose to 288 IU (FIG. 11).

King Oyster spent substrates pressed of excess water and treated with pulsed UV light before and after air drying (FIG. 12) showed slightly higher Vitamin D₂ content when treated wet (9040 IU compared to 6820 IU at 16 pulses). The opposite effect was seen with Maitake spent substrate (FIG. 13). The undried substrate showed less conversion after 8 pulses (1810 IU compared to 3400 IU).

Pulsed UV technology has been shown to be a more practical method of UV irradiation of mushrooms for the mushroom industry than previous methods due to the shorter amount of time needed for exposure to achieve high amounts of Vitamin D₂. The UV-B bulb used in this study was found to be highly effective in converting ergosterol to Vitamin D₂ and would appear to be more practical than UV-C bulbs for commercial use since there would be no generation of ozone that could compromise worker safety.

The ergothioneine content of mushrooms in both fresh and powder form did not appear to change much with pulsed UV treatment. These findings show that it is possible to produce mushrooms that contain high levels of selenium, Vitamin D₂ and ergothioneine.

### Example 2

An experiment was conducted to determine if pulsed UV light treatment employed to enhance the Vitamin D₂ levels could have any negative effects on other nutritionally valuable components like the unique antioxidant L-ergothioneine. Sliced white button mushrooms were exposed to 0, 20, 30, 40 and 50 seconds of pulsed UV light as described above. The results (FIG. 14) demonstrate that Vitamin D₂ levels increased significantly with increasing time of exposure but L-ergothioneine levels were relatively unchanged. These data indicate that mushrooms can be enriched with Vitamin D₂ using pulsed UV light and high ergothioneine levels are retained.

### Example 3

### Experiment

### Effect of Agaricus blazei (1-4) on the Survival Rate of Drosophila melanogaster Fed a Nutritionally Deficient Diet, at Room Temperature (22° C.)

### Samples:

Agaricus blazei (no UV treatment): 1.6 g Vitamin D2/g, dry weight
Two pulses of UV B light: 241.0 g Vitamin D2/g, dry weight
Plain yeast paste base as control
   Prepare vials containing 5.0 ml 1% Agarose medium.
   Prepare yeast paste containing 3% w/w concentration of the two samples
      Preparation of yeast paste: 10.0 gm yeast powder+300 mg of the sample (3%). Grind it in a pestle and mortar. Grind extremely well. Transfer the powder to a small petri dish and add water, mix very well into a paste. Mix well to homogeneity
Apply an equal portion of this paste to the side of the Agarose vials close to the agarose bed.
Collect fresh wild type Canton-S males and females. Age them for 1-2 days. Transfer 3 males and 3 females into each of the agarose vials with yeast paste containing the drug or test substance(s) (6 flies per vial). 15 vials are to be used per sample.
The experiment to be conducted at 22 degrees C. temperature.
Flies to be transferred once in 2 days and the number of flies surviving at each transfer to be recorded.

Drosophila is a model organism with an experimental history of over 100 years. It has a life cycle (embryo to adult) of about 12 days at 22° C. and 9 days at 25° C. The adults live for about 85 days at 22° C. and 60 days at 25° C. under laboratory conditions. It has 3 major chromosomes.

Drosophila and human development are homologous processes. They utilize closely related genes working in conserved regulatory networks. Unlike humans, Drosophila can be genetically manipulated. As a result, most of what we know about the molecular basis of animal development has come from studies of model systems such as Drosophila.

Drosophila has nearly all the important genes that vertebrates including humans have. Not only the genes are conserved but the pathways regulated by these genes are also conserved.

A reliable model using Drosophila as a system to evaluate the effect of a compound for survival on nutritionally deficient diet has been developed by Dr. Krishna Bhat. This method was used to evaluate the effects of A. blazei with and without Vitamin D enrichment on survival at nutritionally deficient diet.

FIG. 17 shows the results.

While preferred embodiments of the present invention have been shown and described, it will be understood by those skilled in the art that various modifications can be made without varying from the scope of the invention.

### Example 4

### Oxidative Stress Experiment

### Experiment

### Testing the Effect of Agaricus blazei (A. blazei) without Enrichment, A. blazei with Vitamin D2 Enrichment, Pure Vitamin D2 and Control (Vehicle for the Delivery) on the Survival Rate of Drosophila melanogaster Under Paraquat-Induced Oxidative Stress Condition

Materials Tested: A. blazei without enrichment, A. blazei with vitamin D2 enrichment, pure vitamin D2 and control (yeast paste—vehicle for the delivery)
   Chemical to induce oxidative stress: Paraquat (10 mM concentration) (Sigma Aldrich). Paraquat is the trade name for N,N′-dimethyl-4,4′-bipyridinium dichloride, a widely used herbicides. Paraquat, a viologen, is quick-acting and non-selective, killing green plant tissue on contact. It is also toxic to human beings when swallowed. This is the most standard chemical used in experimental induction of oxidative stress using the Drosophila model system. It catalyzes the formation of reactive oxygen species (ROS). Paraquat will undergo redox cycling in vivo, gets reduced by an electron donor such NADPH, before being oxidized by an electron receptor such as dioxygen to produce superoxide, a major ROS.
   1. Vials containing 10 mM Paraquat (from Sigma Aldrich) in 5 ml of 1.2% Low melting point Agarose medium were prepared.
   2. A strip of half moist filter paper was inserted in the medium (with the wet end in).
   3. Yeast paste containing 1% concentration (w/w) of the various test materials (see above) mixed to homogeneity was prepared. Yeast paste without drug was used as control.
   4. Uniform aliquot (˜300 mg) of yeast paste with or without the test material) was applied to vials in such a way that yeast paste was on the glass surface and covered the dry end (top) of the filter paper strip.
   5. Freshly enclosed wild type isogenized Canton-S males and females were collected and starved on 1% agar medium for 5-6 hours. Four males and females were transferred to the vial containing 10 mM paraquat in LMP agarose medium and yeast paste with +/− test material (8 flies per vial). 6 vials were used per experiment.
   6. Vials with flies were placed horizontally in a tray. The experiment was conducted at 25 degrees C. temperature.
   7. Flies were transferred once in 2 days and the number of flies surviving at each transfer was recorded.
      Results: Over a period of 10 days, flies fed yeast paste containing A. blazei with vitamin D2 enrichment showed marked and significant survivability under Paraquat-induced oxidative stress condition compared to the control yeast paste alone (54%+/−10% versus 23%+/−8%), yeast paste containing A. blazei without the vit D2 enrichment (54%+/−10% versus 27%+/−8%), and yeast paste containing pure vitamin D2 (54%+/−10% versus 13%+/−3%). Vitamin D2 in its pure form had a deleterious effect on the survival and therefore seems to aggravate the oxidative stress. These results are shown in FIG. 18.
      Conclusion: These results show that a combination of naturally induced Vitamin D2 together with the components of A. blazei has the highest potential and activity to suppress the oxidative stress from Paraquat. Single nutrient or pure vitamin D2 does not have this activity. These findings show a new/novel use for A. blazei enriched with vitamin D2 for suppressing oxidative stress and associated biologic death.

### Example 5

### Title

### A. blazei Enriched with Vitamin D2 Significantly Enhances the Survival and Life Span of Alzheimer's Disease (AD) Model in Drosophila

1) Type of Model (with Specific Drosophila Model of Neurodegeneration, with References)

We used the targeted over/ectopic expression of APP in the brain using a UAS promoter driven APP transgene, induced by a specific GAL4 trans-driver in the brain of a Drosophila model system.

Below is a reference for such over-expression of APP in the Drosophila model system. and the combination gives a fully penetrant AD with limited life-span.

β-Amyloid peptides and amyloid precursor protein (APP) play a deterministic role in Alzheimer's disease (AD). In Drosophila, the targeted expression of the key genes of AD, APP, causes generation of β-amyloid plaques and age-dependent neurodegeneration as well as progression to semilethality, a shortened life span; genetic manipulations or pharmacological treatments with secretase inhibitors influenced the activity of the APP-processing proteases and modulated the severity of the phenotypes (GREEVE I., et al., 2004; The Journal of neuroscience 24, 3899-3906). We used a specific GAL4 driver that induces the APP gene in the central brain area at high levels (see above) and results in a fully penetrant lethality within a 2-3 weeks period. When these AD flies are given A. blazei enriched with vitamin D2, the survival rate was increased nearly double that of the control or A. blazei without any enrichment (FIG. 19). Treating AD flies with pure vitamin D2 or vitamin D3 had no such effect. These results indicate that components in A. blazei, in combination with UV-enriched natural vitamin D2 has significant benefit against the AD disease.

The AD strain lives only for a few days after their eclosion (birth) as opposed to 65 days or more for wild type normal strains. We determined the extension of life span in the mutant strain for each test compound.

While preferred embodiments of the present invention have been shown and described, it will be understood by those skilled in the art that various modifications can be made without varying from the scope of the invention.

### Example 6

A series of experiments were run according to the methods earlier reported on Paraquat induced oxidative stress and Drosophila Alzheimer's disease flies.

We used the targeted over/ectopic expression of APP in the brain using a UAS promoter driven APP transgene, induced by a specific GAL4 trans-driver in the brain of Drosophila model system.

β-Amyloid peptides and amyloid precursor protein (APP) play a deterministic role in Alzheimer's disease (AD). In Drosophila, the targeted expression of the key genes of AD, APP, causes generation of β-amyloid plaques and age-dependent neurodegeneration as well as to semilethality, a shortened life span; genetic manipulations or pharmacological treatments with secretase inhibitors influenced the activity of the APP-processing proteases and modulated the severity of the phenotypes (GREEVE I, et al., 2004; The Journal of neuroscience 24, 3899-3906)”

Since we used a strong GAL4 inducer to activate the hAPP, a significant lethality occurred between one to two weeks after eclosion as opposed to 65 days or more for wild type normal strains. We determined the extension of life span in the mutant strain for each test compound as detailed below.

### Procedure:

Freshly eclosed virgin females from UAS-hAPP strain and males from 408-GAL4 strain were collected and mated in bottles containing cornmeal agar media. Flies were allowed to lay eggs for 3-4 days at 25 degrees C. temperature. Then, the parent flies were transferred to fresh media. The bottles containing eggs from the cross were transferred to 18 degrees C. chamber and allowed to grow until eclosion. Freshly eclosed (virgin) heterozygous (F1) male and female progeny from the cross were collected separately and starved for 5-6 hours in vials containing 1% agar media.

In the mean time, yeast paste containing required concentration of compound was prepared. For mushroom powder, 1% w/w concentration was used. For vitamin D2 and VitD3, 75 mg/10 gm yeast (or 0.75% w/w) concentration was used. Required quantity of both yeast and compound was weighed and finely ground in a pestle and mortar. The finely ground powder was transferred to a small beaker and appropriate quantity of water was added and mixed very well to make a homogeneous paste.

About 300 mg aliquot of yeast paste with/without compound was uniformly applied to the wall of the vial and touching media. Moist filter paper strip was placed inside the vial to maintain humidity. After 5-6 hours of starvation, four males and four females (UAS-hAPP; 408-GAL4) were transferred to each vial containing 1% agar medium with yeast paste (plus or minus compound). These flies were transferred to fresh vials containing same 1% agar medium with yeast paste (plus or minus compound) on every alternate day. This experiment was conducted at 25 degrees C. temperature and the vials were scored for surviving/dead flies at every transfer. Graphs were plotted using the mean percentage survival on alternate day for the treated versus non-treated flies.

The results show that naturally enriched Vitamin D2 mushrooms have the ability to increase biologic survival and nutritionally prevent biologic death as compared to the same unenriched mushroom. The enriched mushrooms further resulted in increased survival when compared to vitamin D2 and vitamin D3 alone. Vitamin D3 actually decreased survival. Agaricus blazei enriched mushrooms had better long term survival than Agaricus bisporus, but both had better effects that non-enriched mushrooms. The results are discussed further below and are shown in FIGS. 19-25.

FIG. 19 is a graph showing Drosophila survival under oxidative stress with A. blazei naturally enriched with vitamin D2. The results show that the enriched mushrooms significantly enhance survival.

FIG. 20 is a graph showing Drosophila survival and prevention of death under Paraquat induced oxidative stress with A. blazei naturally enriched with vitamin D2. The results show that the enriched mushrooms significantly enhance survival and decrease biologic death.

FIG. 21 is a graph showing the Paraquat induced oxidative stress survival with vitamin D3 treatment. The results indicate that vitamin D3 does not prevent biologic death.

FIG. 22 is a graph showing A. blazei enriched vitamin D2 and the survival rate of Drosophila Alzheimer's disease flies. The results show that the enriched mushrooms increase survival in an Alzheimer's disease model.

FIG. 23 is a graph showing that vitamin D2 alone only marginally increases survival of Drosophila Alzheimer's disease flies.

FIG. 24 is a graph showing the effects of vitamin D3 on the survival of Drosophila Alzheimer's disease flies. The results show that vitamin D3 actually DECREASES survival of the flies.

FIG. 25 is a graph showing the effects of added vitamin D2 and D3 compared with the enriched mushrooms on the survival of Drosophila Alzheimer's disease flies. The results show that the natural Vitamin D enhanced mushrooms have the greatest increase in survival.

What is claimed is:

1. A method of increasing longevity and tolerance to oxidative stress in animals comprising:
administering to said animal a naturally enhanced, filamentous fungi, tissue, substrate, spent substrate or component thereof, with increased levels of Vitamin D, wherein upon administration of the same, longevity is increased.

2. The method of claim 1 wherein said Vitamin D is Vitamin D₂.

3. The method of claim 1 wherein said filmentous fungi is a mushroom.

4. The method of claim 3 wherein said mushroom is of a species selected from the group consisting of: Agaricus bisporus, Agaricus blazei, Lentinula edodes, Pleurotus ostreatus.

5. The method of claim 4 wherein said mushroom is enriched by pulsed UV irradiation.

6. The method of claim 4 wherein said mushroom's ergothioneine content remains unchanged after enrichment.

7. The method of claim 3 wherein said fungi is in powder form.

8. The method of claim 2 wherein said Vitamin D₂ content is increased to about 800% of the daily recommended value of Vitamin D.

9. The method of claim 1 wherein said filamentous fungi does not have discoloration from the enrichment.

10. The method of claim 1 wherein said tissue is mycelium.

11. The method of claim 1 wherein said substrate is air dried.

12. A nutritional product for increasing longevity and tolerance to oxidative stress in animals comprising a UV irradiated, filamentous fungi, tissue, substrate or component thereof with higher levels of Vitamin D than a non-irradiated product.

13. A method of increasing resistance to oxidative stress and associated disease states such as Alzheimer's disease in animals comprising:
administering to said animal an effective amount of a filamentous fungi that has been naturally enriched in Vitamin D₂.

14. The method of claim 13 wherein said enrichment is from UV treatment.

15. The method of claim 13 wherein said enrichment is from pulsed UV irradiation.

16. A method of increasing longevity in animals suffering from a nutritional deficit comprising:
administering to said animal a pulsed UV irradiated, filamentous fungi, tissue, substrate, spent substrate or component thereof, with increased levels of Vitamin D, wherein upon administration of the same, longevity is increased.

17. The method of claim 16 wherein said Vitamin D is Vitamin D₂.

18. The method of claim 16 wherein said filamentous fungi is a mushroom.

19. The method of claim 18 wherein said mushroom is of a species selected from the group consisting of: Agaricus bisporus, Agaricus blazei, Lentinula edodes, Pleurotus ostreatus.

20. The method of claim 18 wherein said mushroom is selenium enriched.

21. The method of claim 18 wherein said mushroom's ergothioneine content remains unchanged after UV treatment.

22. The method of claim 18 wherein said fungi is in powder form.

23. The method of claim 17 wherein said vitamin D₂ content is increased to about 800% of the daily recommended value of said vitamin.

24. The method of claim 16 wherein said filamentous fungi does not have discoloration from the UV treatment.

25. The method of claim 16 wherein said tissue is mycelium.

26. The method of claim 16 wherein said substrate is air dried.

27. A method of treating a disease state associated with increased amyloid precursor protein, oxidative stress, or production of free radicals such as Alzheimer's disease and/or taupathies, and similar neurodegenerative diseases in animals comprising:
administering to said animal with said disease state a pulsed UV irradiated, filamentous fungi, tissue, substrate, spent substrate or component thereof, with increased levels of vitamin D2, wherein upon administration of the same, survivability of said animal is increased when compared to an animal with such disease state without such treatment.

28. The method of claim 27 wherein said filmentous fungi is a mushroom.

29. The method of claim 28 wherein said mushroom is of a species selected from the group consisting of: Agaricus blazei, Agaricus bisporus, Lentinula edodes, Pleurotus ostreatus.

30. The method of claim 29 wherein said mushroom is Agaricus blazei.

31. The method of claim 30 wherein said mushroom is selenium enriched.

32. The method of claim 30 wherein said mushroom's ergothioneine content remains unchanged after UV treatment.

33. The method of claim 27 wherein said fungi is in powder form.

34. The method of claim 28 wherein said vitamin D2 content is increased to about 800% of the daily recommended value of said vitamin.

35. The method of claim 27 wherein said filamentous fungi does not have discoloration from the UV treatment.

36. The method of claim 27 wherein said tissue is mycelium.

37. The method of claim 27 wherein said substrate is air dried.

38. A pharmaceutical composition for treating a disease state associated with increased amyloid precursor protein comprising a UV irradiated, Agaricus fungi, tissue, substrate or component thereof with higher levels of vitamin D2 than a non-irradiated product and a carrier.

39. The pharmaceutical composition of claim 38 wherein said product comprises Agaricus blazei.

40. The pharmaceutical composition of claim 39 wherein said Agaricus blazei comprises higher levels of vitamin D2 than a non-irradiated product.

41. The pharmaceutical composition of claim 38 wherein said Agaricus fungi is in powder form.
